(19) United States
(12) Patent Application Publication
(10) Pub. No.: US 2011/0028864 A1
(43) Pub. Date: Feb. 3, 2011

(54) Uterine sound device

(76) Inventors: Margaret Doubrava, Las Cruces, NM (US); Donna F. Bateman, Las Cruces, NM (US)

Correspondence Address: Donna F. Bateman, 1642 High Street, Las Cruces, NM, 88011, US

(21) Appl. No.: 12/462,262

(22) Filed: Jul. 31, 2009

(51) Int. Cl. A61B 5/107

(52) U.S. Cl. 600/591

(57) ABSTRACT

A uterine sound device includes a first component having a handle and a stem, and a slidable member positioned around the stem. An inner wall of the slidable member fits around the stem such that the slidable member remains at a fixed position on the stem unless a predetermined amount of force is applied to the slidable member longitudinally along the stem. The stem includes graduated markings to indicate units of measure that are preferably formed of a non-leachable, biocompatible ink printed on the stem. In use, the sound stem is inserted past the cervix with the slidable member remaining on the outside of the cervix with insertion pressure causing the slidable member to slide along the stem toward the handle. The position of the slidable member according to the indicia indicates a depth into the uterus that the stem has reached.

### FIELD OF THE INVENTION

This invention relates generally to gynecological medical devices and, more specifically, to devices for measuring the depth and/or course of a uterine cavity.

### BACKGROUND OF THE INVENTION

In the gynecological field, it is sometimes necessary for a physician to measure the internal depth and/or course of a uterine cavity. For instance, such measurement may be necessary to prevent the accidental perforation of the uterus during a subsequent surgical procedure. In other instances, such measurements may be performed both before and after a surgical procedure to ensure that the uterus has properly contracted upon completion of the procedure.

A disposable uterine sound is disclosed in U.S. Pat. No. 5,109,869 that relates to a disposable uterine sound with an ink-marked shaft. A disadvantage to the invention disclosed in this patent is that the ink markings are extremely difficult to read while the sound is inside the patient and it is difficult to determine how deeply the sound has been inserted to acquire an accurate reading once removed from the patient. This defeats the purpose of the device which is to accurately measure the depth and course of the uterus.

A uterine sound is also disclosed in U.S. Pat. No. 4,489,732 that relates to a uterine sound with a moveable index point where pressure from being pushed against the cervix allows an accurate reading on a protected gauge. There are several disadvantages to the uterine sound disclosed in this patent. It has a relatively high cost of production. It requires that an amount of pressure be applied to compress a spring which otherwise holds an index point in place that may make it difficult for a physician to determine by feel when the maximum depth of a uterine cavity has been reached thereby facilitating accidental perforation. The device is also far more complex than required. The unnecessarily complex nature of this uterine sound can lead to a greater possibility of something going wrong during a measurement procedure.

Other previously existing uterine sounds often have a stem or shaft marked by indentations. This indented outer surface of the device can induce unnecessary trauma during insertion and removal, sometimes collecting blood which has been used to allow the physician a point of reference on the surface of the sound to better determine the depth of the uterus once the device is removed. An obvious disadvantage to this type of device is the unnecessary trauma induced on the patient by the device.

### SUMMARY OF THE INVENTION

A uterine sound device for measuring the depth of a uterine cavity in accordance with an embodiment of the invention includes a first component having a handle and a stem, and a slidable member positioned around the stem. An interior wall of the slidable member fits around the stem such that the slidable member remains at a fixed position on the stem unless a predetermined amount of force is applied to the slidable member longitudinally along the stem.

In accordance with one embodiment of this invention, the stem includes graduated markings to indicate units of measure.

In accordance with other embodiments of this invention, the graduated markings are formed of a non-leachable, biocompatible ink printed on the stem.

In accordance with still further embodiments of this invention, the slidable member is a circular ring.

In accordance with yet other embodiments of this invention, the slidable member is formed of a nontoxic plastic material.

In accordance with still another embodiment of this invention, the slidable member is an O-ring.

In accordance with still further embodiments of this invention, the stem is a semi-flexible stem having a predetermined flexibility such that the stem is rigid enough to penetrate a cervix yet flexible enough to bend about a contoured region of a uterus such that a course of the uterus may be measured.

In accordance with yet another embodiment of this invention, the stem has a rounded tip.

In accordance with additional embodiments of this invention, the stem has a blunt tip.

In accordance with further embodiments of this invention, the handle and the stem are formed of a material capable of undergoing a sterilization process so that they can be provided to a user in a sterile form.

In accordance with still further embodiments of this invention, the slidable member is also formed of a material capable of undergoing a sterilization process so that it can be provided to the user in a sterile form.

In accordance with additional embodiments of this invention, the first component is a single integral piece with a handle portion and a stem portion.

In accordance with yet other embodiments of this invention, the stem of the first component is a separate piece attached to the handle.

In accordance with other embodiments of this invention, the stem is permanently attached to the handle in a non-removable manner.

In accordance with still other embodiments of this invention, the handle is structured to define a recessed portion for receiving a thumb of a user to provide an improved grip.

In accordance with still further embodiments of this invention, the stem is curved a predetermined amount in a region of the stem opposite the handle.

In accordance with yet other embodiments of this invention, a method of determining a uterine depth includes holding a vagina open with a speculum, inserting the stem and slidable member into the vagina, inserting the rounded tip of the stem through a cervix into a uterus, pushing the stem through the slidable member and into the uterus as the slidable member is held back from entering the uterus by the cervix, removing the uterine sound device from the uterus and vagina, and determining a uterine depth by looking at a location of the slidable member along the stem as shown by the graduated markings.

These and other embodiments of the invention will be described in further detail below.

### BRIEF DESCRIPTION OF THE DRAWINGS

Preferred and alternative embodiments of the present invention are described in detail below with reference to the following drawings:

FIG. 1 is a side view of a uterine sound formed in accordance with an embodiment of the invention;

FIG. 2 is a top view of the uterine sound shown in FIG. 1, with a front portion of the uterine sound being inserted through a cervix into a uterus;

FIG. 3 is a perspective view of a portion of the uterine sound shown in FIGS. 1 and 2 showing additional detail for a slidable ring and markings on a stem of the uterine sound;

FIG. 4 is a cross-sectional end view of the stem of the uterine sound; and

FIG. 5 is a cross-sectional view of the slidable ring.

### DETAILED DESCRIPTION OF THE PREFERRED EMBODIMENT

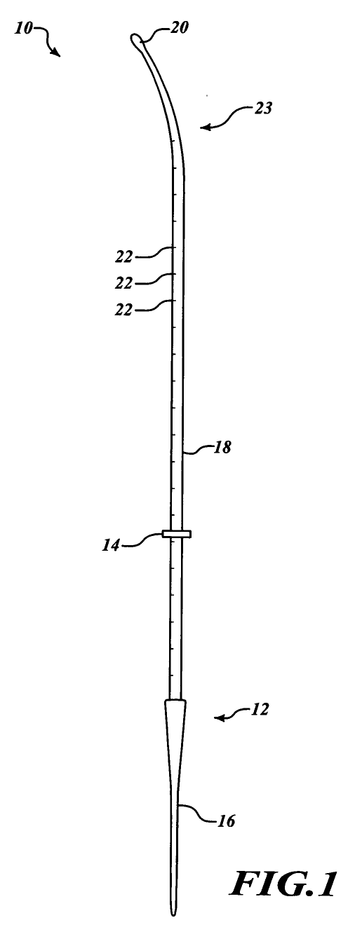

Referring now to FIG. 1, which is a side view of a uterine sound 10 in accordance with an embodiment of the invention. The uterine sound 10 includes an elongated first element 12 and a second element 14 that is slidably connected to the first element 12. In the example embodiment shown, the elongated first element 12 is structured as a single piece having a handle 16 and a stem 18 (also referred to as a shaft or probe). However, in other embodiments, the handle 16 and the stem 18 may be separate parts that have been attached together, preferably permanently, to form the first elongated element 12. The first element 12 is preferably formed of a plastic polymer material, but may also be formed of a metal, or other materials in some embodiments. The first element may also be formed of more than one layer and/or type of material in some embodiments.

The second element 14 is a non-toxic plastic ring around the stem 18. The ring is an O-ring in some embodiments. Although the second element 14 is a non-toxic plastic ring in the embodiment shown, the second element 14 may also be formed of other materials such as a silicone based material, for example and/or may have a shape other than a circular ring in other embodiments. In an example embodiment, the uterine sound 10 is intended to be a single-use disposable device. However, in other embodiments, one or more of the components of the uterine sound 10 may be made of materials capable of undergoing a sterilization process such as standard autoclaving procedures. For example, the first element 12 may be made of a material such as a Teflon® based thermostable plastic and/or the second element 14 may be made of a material such as a silicone based thermostable plastic.

The stem 18 preferably has a rounded tip 20 that minimizes the chance of accidental perforation of the uterus while providing a means to ensure that the second element 14 is properly positioned posterior to this point prior to insertion. A plurality of marked indicia 22 on the stem 18 allow for the measurement of the depth of the uterine cavity via the final position of the second element 14 in relationship to these indicia 22 on the stem 18 after removal of the uterine sound 10. The indicia are marked in centimeter (cm) increments and fractions thereof in the example embodiment shown (fractional markings not shown for clarity), but other incremental distances may be used in other embodiments. Not all of the indicia 22 are indicated with reference numerals for clarity. The handle 16 is sized with a predetermined width and shape such that the second element 14 is prevented from sliding onto the handle 16 and coming in contact with a user of the uterine sound 10. In the embodiment shown, the stem 18 is curved upward by a predetermined amount in a region 23 toward the tip 20 opposite the handle 16. However, the stem 18 may not be curved in this manner in other embodiments. In an embodiment, the stem 18 is made of a flexible material that conforms to a course of a uterus during insertion and retains this conformation upon withdrawal thereby allowing the course of the uterus to be measured in addition to its depth. In an embodiment, the flexible material is a semi-flexible material having a predetermined flexibility such that the stem 18 is rigid enough to penetrate a cervix yet flexible enough to bend about a contoured region of a uterus.

In one embodiment of this invention, the uterine sound 10 is a two-piece device consisting of only the first elongated element 12 formed as a single integral piece with the handle 16 and stem 18 with the rounded tip 20, and the second element 14. This allows for a decreased expense of production, simplifies the complexity of the uterine sound 10, and decreases chances of accidental perforation during use, while retaining desired functionality.

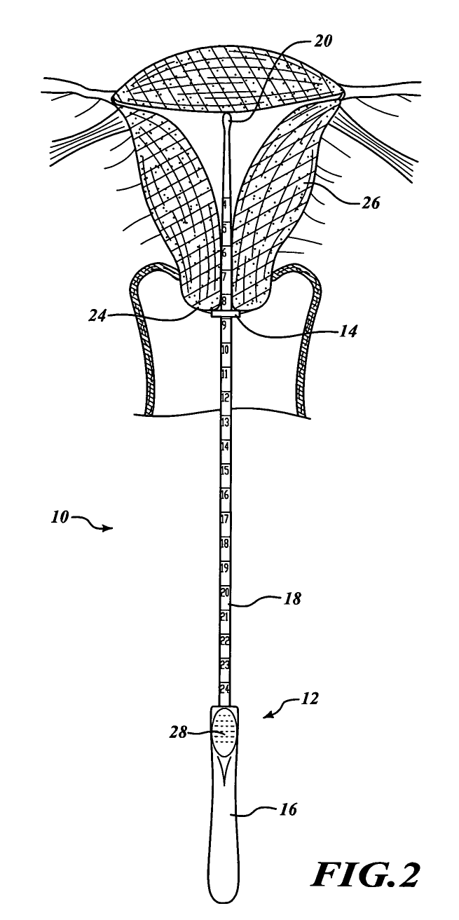

FIG. 2 is a top view of the uterine sound 10 being inserted through a cervix 24 into a uterus 26. This demonstrates the function of the second element 14 which abuts the entrance to the cervical canal and is pushed back along the stem 18 by pressure from the cervix 24 during insertion, coming to a final stopping place of rest when the rounded tip 20 of the sound 10 reaches the full depth of the uterus 26 and is stopped by the uterine wall. When the sound 10 is extracted, the location of the second element 14 on the stem 18 in relationship to the marked indicia 22 allows for the accurate determination of the depth of the uterus 26. The handle 16 defines a recessed portion 28 for receiving a thumb of a user to provide an improved grip.

In use, a user such as a doctor positions the second element 14 on the stem 18 near the tip 20 and inserts the tip 20 of the uterine sound 10 through the cervical os and into the cervical canal. The second element 14 rests on the outside of the cervix 24. The user slowly advances the uterine sound 10 into the uterine cavity until the tip 20 meets the back (fundus) of the uterus 26. The second element 14 remains at rest on the outside of the cervix 24 during insertion of the uterine sound 10 into the uterine cavity. The second element 14 remains in place on the stem 18 at its farthest measurement from the back of the uterus 26. The user slowly pulls the uterine sound 10 out of the uterus 26 and cervix 24 and visualizes the depth of the uterus 26 by looking at the position of the second element 14 on the stem 18, with the measurement being indicated by the indicia 22 as measured from the tip 20 of the uterine sound 10 to a face of the second element 14 nearest the tip 20. There is sufficient friction between the second element 14 and the stem 18 so that the second element 18 will remain in position on the stem 18 as the uterine sound 10 is withdrawn so that the final position of the second element 14 on the stem 18, in reference to the graduated marks provided by the indicia 22 give an accurate indication of the depth of the uterus 26.

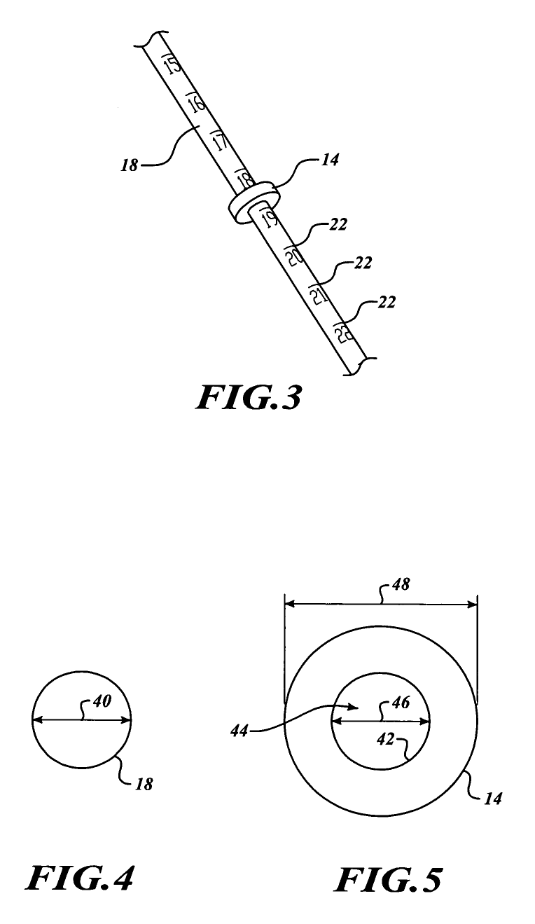

FIG. 3 is a perspective view of a portion of the uterine sound 10 shown in FIGS. 1 and 2 showing additional detail for the second element 14 in relation to the stem 18 and the indicia 22. The indicia 22 are preferably formed of a non-leachable, biocompatible ink printed on the stem 18. This helps to minimize trauma to the patient in comparison to indicia formed of indentations in the stem 18.

FIG. 4 is a cross-sectional end view of the stem 18 of the uterine sound 10. In an embodiment of this invention, the stem 18 has a predetermined diameter 40 that is approximately the same at least in a region of the stem 18 where the second element 14 would be positioned to indicate predetermined typical minimum and maximum uterine depths. In one embodiment, the diameter 40 is approximately 4 millimeters (mm), but other diameters can also be used in other embodiments.

FIG. 5 is a cross-sectional view of the second element 14. The second element 14 has an inner wall 42 and defines a hollow interior portion 44 that the stem 18 fits within. In an example embodiment, the second element 14 has an inner diameter 46 that is slightly smaller by a predetermined amount than the diameter 40 of the stem as shown in FIG. 4 when the second element 14 is not positioned on the stem 18. After the second element 14 is positioned on the stem 18, the second element 14 stretches to accommodate the diameter 40 of the stem 18 within the hollow interior portion 44. The inner diameter 46 is sized such that the second element 14 remains at a fixed position on the stem 18 unless a predetermined amount of force is applied to the second member 14 longitudinally along the stem 18. Generally, the predetermined amount of force will be low enough to allow the stem 18 to slide through the second element 14 when being inserted into a uterus as the second element is held back by a cervix and will be high enough to keep the second element 14 in place on the stem 18 as the uterine sound 10 is removed from a uterus and vagina.

The second element 14 has a predetermined outer diameter 48. The outer diameter 48 is sized such that the second element 14 will fit though a vagina held open by a speculum yet will not fit through a cervical os in a typical procedure, but will be held back from entering the uterus by the cervix.

It is recognized that changes or modification may be made to the embodiments depicted and described herein without departing from the scope of the invention. Therefore, it is intended that the foregoing drawings and description shall be interpreted as an illustration of the invention and not as limitations upon the invention.

While the preferred embodiment of the invention has been illustrated and described, as noted above, many changes can be made without departing from the spirit and scope of the invention. For example, non-circular cross-sectional profiles of the stem and/or inner and outer diameters of the second element may be used in some embodiments. The resistance to movement between the second element 14 and the first elongated element 12 may be achieved in other manners as well, such as by having the second element 14 be formed of a rigid material with the first elongated element 12 being formed of one or more materials that allow the diameter 40 of the stem 18 to be deformed slightly such that a desired resistance to movement is achieved between the rigid second element 14 and the first elongated element 12. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A uterine sound device for measuring the depth of a uterine cavity, the sound device comprising:
a first component having a handle and a stem; and
a slidable member positioned around the stem,
wherein the stem includes graduated markings to indicate a unit of measure, the graduated markings printed on the stem, and wherein an interior wall of the slidable member fits around the stem such that the slidable member remains at a fixed position on the stem unless a predetermined amount of force is applied to the slidable member longitudinally along the stem.

2. The uterine sound device of claim 1, wherein the slidable member is a circular ring.

3. The uterine sound device of claim 2, wherein the circular ring is formed of a non-toxic plastic material.

4. The uterine sound device of claim 2, wherein the circular ring is an O-ring.

5. The uterine sound device of claim 1, wherein the stem is a semi-flexible stem having a predetermined flexibility such that the stem is rigid enough to penetrate a cervix yet flexible enough to bend about a contoured region of a uterus,
whereby a course of the uterus may be measured.

6. The uterine sound device of claim 1, wherein the stem has a rounded tip.

7. The uterine sound device of claim 1, wherein the stem has a blunt tip.

8. The uterine sound device of claim 1, wherein the handle and the stem are formed of a material capable of undergoing a standard autoclaving sterilization process.

9. The uterine sound device of claim 8, wherein the slidable member is also formed of a material capable of undergoing a standard autoclaving sterilization process.

10. The uterine sound device of claim 1, wherein the first component is a single integral piece with a handle portion and a stem portion.

11. The uterine sound device of claim 1, wherein the stem has a first diameter and the slidable member has an inner diameter smaller than the first diameter when the slidable member is removed from the stem.

12. The uterine sound device of claim 11, wherein the slidable member is formed of a stretchable material.

13. The uterine sound device of claim 1, wherein the handle is structured to define a recessed portion for receiving a thumb of a user to provide an improved grip.

14. The uterine sound device of claim 1, wherein the stem is curved a predetermined amount in a region of the stem opposite the handle.

15. The uterine sound device of claim 1, wherein the graduated markings are formed of a non-leachable, biocompatible ink printed on the stem.

16. A uterine sound device for measuring a depth of a uterine cavity, the sound device comprising:
a first component having a handle and a stem; and
a circular ring positioned around the stem,
wherein the stem includes graduated markings to indicate a unit of measure, the graduated markings printed on the stem, wherein the stem has a rounded tip, and wherein an interior wall of the circular ring fits around the stem such that the ring remains at a fixed position on the stem unless a predetermined amount of force is applied to the ring longitudinally along the stem.

17. The uterine sound device of claim 16, wherein the first component is a single integral piece with a handle portion and a stem portion.

18. The uterine sound device of claim 17, wherein the handle is structured to define a recessed portion for receiving a thumb of a user to provide an improved grip.

19. The uterine sound device of claim 18, wherein the stem is a semi-flexible stem having a predetermined flexibility such that the stem is rigid enough to penetrate a cervix yet flexible enough to bend about a contoured region of a uterus,
whereby a course of the uterus may be measured.

20. The uterine sound device of claim 16, wherein the graduated markings are formed of a non-leachable, biocompatible ink printed on the stem.

21. A method of determining a uterine depth using a uterine sound device having a first component with a handle and a stem, the stem having a rounded tip and graduated markings printed on the stem, the uterine sound device also having a slidable member positioned around the stem, wherein an interior wall of the slidable member fits around the stem such that the slidable member remains at a fixed position on the stem unless a predetermined amount of force is applied to the slidable member longitudinally along the stem, the method comprising:
holding a vagina open with a speculum;
inserting the stem and slidable member into the vagina;
inserting the rounded tip of the stem through a cervix into a uterus;
pushing the stem through the slidable member and into the uterus as the slidable member is held back from entering the uterus by the cervix;
removing the uterine sound device from the uterus and the vagina; and
determining a uterine depth by looking at a location of the slidable member along the stem as shown by the graduated markings.

22. A uterine sound device for measuring the depth of a uterine cavity, the sound device consisting of:
a first component having a handle and a stem formed as a single integral piece; and
a slidable circular ring positioned around the stem,
wherein the stem includes graduated markings to indicate a unit of measure, the graduated markings formed of a non-leachable, biocompatible ink printed on the stem, and wherein an interior wall of the slidable circular ring fits around the stem such that the slidable circular ring remains at a fixed position on the stem unless a predetermined amount of force is applied to the slidable circular ring longitudinally along the stem.

23. The uterine sound device of claim 22, wherein the stem has a first diameter and the slidable circular ring has an inner diameter smaller than the first diameter when the slidable member is removed from the stem.
